(19) United States
(12) Patent Application Publication
(10) Pub. No.: US 2013/0303936 A1
(43) Pub. Date: Nov. 14, 2013

(54) Advanced Ovulation Predictor Device

(71) Applicant: Russell L. Case, JR., Oviedo, FL (US)

(72) Inventor: Russell L. Case, JR., Oviedo, FL (US)

(21) Appl. No.: 13/892,996

(22) Filed: May 13, 2013

Related U.S. Application Data
(60) Provisional application No. 61/645,689, filed on May 11, 2012.

(51) Int. Cl. A61B 10/00

(52) U.S. Cl. CPC A61B 10/0012
USPC 600/551

(57) ABSTRACT

The invention is a hand-held advanced ovulation predictor device, which includes an advanced ovulation predictor device body, an optical subassembly containing one or more hybrid lenses, an electronics assembly with a light source, switch, and battery, and, optionally, a cover. The preferred embodiment of this invention is one in which the viewing axis and the device axis are not collinear or coaxial, the specimen is illuminated with oblique or dark field illumination, the method of transmitting the light is through a light pipe, and one or more lenses are hybrid lenses.

This application claims priority from U.S. provisional patent application Ser. No. 61/645689, filed May 11, 2012.

### FIELD OF INVENTION

The present invention relates to devices for predicting ovulation in animals, in particular to hand held ovulation predictors.

### BACKGROUND

There are many devices for predicting ovulation in animals and the art is well known. In modern culture, with ever increasing demands on a woman's time, she requires an accurate, portable, and discreet device to assist in identification of ovulation and as an aid in family planning. The knowledge gained from daily monitoring of the often unpredictable ovulation cycle is instrumental in assisting the woman to become pregnant or to avoid pregnancy. The present invention is a unique and advanced device used to monitor and track ovulation based on chemical variations in body fluids which occur in a female during ovulation and the associated change in the crystallization of components of those fluids during the ovulation cycle.

As early as 1916 Dr. George N. Papanicolau introduced the vaginal smear to determine its usefulness in studying the female mammalian sex cycle. Over the next 30 years, Dr. Papanicolau studied the phenomena and characteristics of the vaginal smear as it correlated to the ovulation cycle, even experimentally inducing the effects through administration of estrogenic hormones. He identified a secretion that when abundant tends to spread in the form of anastomosing branches with heavily indented leafy projections. The secretion became very typical and conspicuous during the peak of follicular activity and decreased and practically disappeared during other stages of the cycle, during pregnancy, and in inactive states, such as amenorrhea or menopause. Dr. Papanicolau went on to explain that a complete evaluation of the normal sex cycle requires an examination “not only during the follicular and postovulatory stages, but during all phases of the cycle.” These findings were presented before the Chicago Gynecological Society on 20 Apr. 1945. He went on to publish his findings in the American Journal of Obstetrics and Gynecology (Papanicolau, G. N. (1946). A General Survey of the Vaginal Smear and its Use in Research and Diagnosis. American Journal of Obstetrics and Gynecology, 51, 316-324).

The crystallization of cervical mucus was also observed by Dr. Erik Rydberg, a Professor at the University Clinic of Obstetrics and Gynecology, Copenhagen, Denmark, and with the help of Professor Linderstrom-Lang of the Carlsberg Laboratory, also in Copenhagen, Denmark, determined that the crystals were common salt and that the “strange formations” were due to the presence of mucin. (The presence of mucin was later refuted by further investigations and determined that the presence of protein solutions elicited the pattern.) It was also observed that the drying of salt solutions in the presence of proteins developed shapes strikingly like those found in cervical mucus. Furthermore, the same crystal formations were found in dried saliva, especially in the days close to ovulation. (Rydberg, E. (1948). Observations on the Crystallization of the Cervical Mucus. Acta Obstetricia et Gynecologica Scandinavica, 28, 172-187).

By 1954 the crystallization pattern was called arborization, referring to the “pal-like” or “fern-like” appearance of the crystals and was widely studied as a diagnosis for ovulation. In one such study, Drs. Bernhard Zondek and Samuel Rozin concluded that the PL (Palm Leaf) test can be used diagnostically for determining the corpus leteum function, associated with the release of the oocyte. (Zondek, B., Rozin, S. (1954). Cervical Mucus Arborization: Its use in the Determination of Corpus Luteum Function. Obstetrics & Gynecology 3, 463-470).

Finally, in 1968, Dr. J. M. Biel Casals observed that at times dried saliva left on a slide to dry adopts on some occasions a branch-like appearance similar to cervical crystallization, and at other times it presents an amorphous appearance. After recording a great number of cases, he concluded that the branch-like pattern accentuates as a woman approaches the day of ovulation and lessens quickly thereafter. He noted that using dried saliva as a test for ovulation was almost unknown in gynecological circles, except in his hospital where providers applied the principal routinely. (Biel Casals, J. M. (1968). Description De Un Nuevo Test De Ovulacion Y Analisis De Su Resultados. Medecina clinica, 50, 385-392).

The Advanced Ovulation Predictor Device relies on the crystallization pattern of common salt (NaCl) in the presence of proteins in solution and that the concentrations of these proteins, in synchronization with the ovulation cycle, determine the intensity of the phenomenon known as “arborization.”

There are a number of devices designed for ovulation prediction.

U.S. Pat. No. 3,582,181 is a pocket microscope comprising a housing having first and second tubular elements coaxially press fitted together. An eyepiece is mounted on the first tubular element and an illuminating means is mounted on the second tubular element. The illuminating means includes a lamp and a battery which is normally maintained out of contact with the lamp. A manually movable actuator member is mounted on the housing end cap for causing relative movement of the battery towards the lamp. An objective means is movably mounted within the housing and is selectively movable axially thereof.

U.S. Pat. No. 4,815,835 describes a microscope as an optical apparatus with a slide lighting system for detecting a fertile period during a woman's menstruation cycle from her saliva and has inner and outer telescopic tubular bodies. The inner tubular body has an optical system of three, fixed lenses for 100 fold magnification of the saliva on a slide. The outer tubular body has, at one free end, a window for receiving the slide and threading for the axial attachment of another light-device tubular body for lighting the slide.

U.S. Pat. No. 5,267,087 is a portable diagnostic apparatus for the examination of biological material, in particular for the self-examination of a specimen containing substances which differ as a function of ovulation. The housing of the apparatus is designed such that the apparatus is of a size which allows the examining apparatus to be carried in the pockets of articles of clothing.

U.S. Pat. No. 5,572,370 describes a compact, self-contained, easy-to-use and readily portable apparatus which can be carried in a woman's purse or pocket for determining the period of maximum fertility. The apparatus includes a hollow housing that sealably contains a small magnifying system, a saliva specimen slide and an internal switch operated illuminating system for illuminating the saliva slide. The small magnifying system is used to closely view patters formed on the specimen slide by the saliva after it has dried and crystallized on the slide. The patterns are then compared with standard comparison patterns to determine the woman's present ability to conceive.

U.S. Pat. No. 5,639,424 deviates from the coaxial tubular style and describes a portable fertility tester for identifying days during the menstrual cycle when a woman is most likely to conceive has a circular disc, with transparent regions indexed to each of the days of the cycle, rotatably mounted within a housing and cover. The housing and cover form a window for exposing a transparent region on the disc for depositing a saliva specimen. An ocular is provided with a magnifying lens for examining the appearance of the saliva which is indicative of fertility, and a light emitting diode is provided for backlighting the specimen during examination. The cover may be removed to allow replacement of the disc for each menstrual cycle.

U.S. Pat. No. 5,837,197 is a fertility analysis and reproductive health system that is applicable to both female and male mammals. In particular the Invention is a portable, handheld, integrated unit which can be manufactured out of plastic. The unit can be disposable for hygienic purposes, or cleaned or sterilized for repeated use as desired.

U.S. Pat. No. 6,582,377 is a high accuracy fertility-no-fertility choice device for women. The device-indicator is based on precise examination of the saliva estrogen and for that purpose is used either linear or circular polarized light, plus quick spectral and contrast analysis. All features of the device are built in a special purpose individual mini microscope.

U.S. Pat. No. 6,793,886 describes a device for the detection of characteristics and parameters of body fluids such as saliva, urine and cervical mucus for the purposes of studying and identifying fertility periods in women, comprising a set of flat plate-shaped supports for samples of said body fluids and a viewer provided with enlargement means, characterized in that each of said flat plate-shape supports for body fluid presents a shallow basin or trap with a convex bottom entirely surrounded by a raised rim, and is equipped with locking fins suitable for coupling with structural elements present on said viewer so that it is irreversibly locked onto the latter in a desired relative position.

U.S. Pat. No. 7,369,331 is an almost identical device when compared to U.S. Pat. No. 5,572,370, but still provides a fixed focus handy ovulation tester, the said ovulation tester comprising an inner casing, having a top and a bottom end; a controllable illuminating assembly located inside the inner casing and near the bottom end of the inner casing and being sealed at the bottom by a bottom face plate and a fixed focus eye piece assembly having a bottom inner portion for placing a biological specimen and a top outer portion for viewing the specimen being removably located at the top end of the inner casing.

U.S. Pat. No. 7,799,275 is a diagnostic test device comprises means for sampling a liquid biological sample and means for reacting the sample with at least one reagent to provide one or more visible indicia. The device also comprises an optical detector for detecting the presence of the one or more indicia. The device also comprises means for causing at least part of the optical detector to move over the one or more indicia.

US Patent Application 2003/0179446 is a portable microscopic visualization tube for determining the ovulation period from saliva having a microscopic lens module, a beam tube, an electric powered light emitting diode mechanism, and a tube cap, and the light emitting diode mechanism includes a button battery seat, characterized in that the inner wall of the beam tube is correspondingly formed into engageable recessing block or protruding block such that the entire light emitting diode mechanism can be withdrawn from the beam tube to replace the button battery within the button battery seat.

US Patent Application 2006/0018043 describes a portable handheld fertility/ovulation tester that uses ambient light, and requires no power. A unique sample holding frame and adjustable lens assembly is inserted into a light chamber in the bottom of the tester. An aperture in the bottom of the chamber is aligned with a microscope lens assembly and is sized to provide ambient light for the microscope lens assembly. The light entering the chamber and its direction are controlled by the size and shape of the aperture, which may also have an optional light gathering lens to increase illumination. The adjustable lens assembly is threaded into a sample plate frame having a transparent sample plate. The adjustable lens assembly is threaded into a sample plate frame having a transparent sample plate. The microscope lens assembly is removably mounted onto the light chamber. The lens assembly is mounted unto the light chamber such that when the fertility ovulation tester is held with the aperture pointed towards an ambient light source, the user can easily observe the sample.

U.S. Patent Application 2011/0282247 is a hand held ovulation predictor device which includes an ovulation predictor device body, an optical subassembly containing one or more aspheric lenses, and electronics assembly, a battery compartment, a light source, and optionally a cover.

Japanese Patent JP 08-068790 describes an ovulation day measuring equipment, convenient to store and carry, by using a microscopic member provided at one end of a tube to enlarge and see through a woman' s saliva. The device was designed to measure an ovulation day, by using a microscopic member that is pulled outside and separated from a tube body. Then, the woman's (examinee) saliva is attached to the outside face of a fluoroscope and dried. The microscopic member is inserted into one end of the tube body. At this time, a snap spring is shrunk and softly inserted therein. A push switch is pushed by a finger to light a lamp so that the enlarged image of the saliva applied to the fluoroscope can be observed by naked eyes through the microscopic member. A control is turned to the right or left to move a lens along the axial direction of the tube body for providing a clear image. If the observed image is formed in a fern shape, the examinee is on the ovulation day.

No currently disclosed device utilizes ergonomics to enhance the viewing comfort of the user. In the case of the tubular coaxial design, the viewing axis and the major axis of the device are aligned. This creates a condition where the user finds the observation and discrimination of the sample difficult and prone to error. Additionally, the user must use two hands to manually actuate the light switch while adjusting the focus of the lens, while in the present invention, the feature of viewing axis and major axis being non-coaxial, complex shape with gripping surfaces, automatic illumination, and focus based on eye relief, allows the operator to use the device with one hand.

No currently disclosed device utilizes light pipe technology for illuminating the sample.

All of the currently available devices utilize bright field illumination of the sample. In bright field illumination, light enters the device along an optical axis either from above or from below the sample. That light is absorbed by the sample, creating the appearance of the sample being darker than the surrounding field of view. In the case of saliva and salt, the sample under observation is transmissive in nature, allowing much of the light to pass through the sample and reduce contrast ratio. Contrast ratio is the effect that allows visualization of the sample.

In the prior art, the sample objective plate is located inside the device, requiring the user to partially disassemble the device, apply a sample, and insert the objective plate into the device and align it with the optical path.

In all but one of the currently available devices, simple convex, concave, planar or spherical lenses are utilized. One device enhances the image by using aspherical lenses. However, in all cases the manufacturing aspects are complex and costly.

Further, the current devices require the user to separately activate the light source or utilize ambient light. Additionally, many of the devices incorporate separately removable covers, which may become lost or damaged.

There is a need for an advanced technology, compact, inexpensive, discreet and portable ovulation predictor device that is ergonomic, has enhanced manufacturability, automatic lighting, enhanced illumination, and eliminates the requirement to focus the lens system.

### SUMMARY OF THE INVENTION

The present invention is a hand-held advanced ovulation predictor device 100, which includes an advanced ovulation predictor device body top 101, an advanced ovulation predictor device body bottom 102, one or more hybrid lenses 201, an objective plate 202, and a light pipe 203. Further, the advanced ovulation predictor device contains an electronics subassembly consisting of a printed circuit board 301, a light emitting diode 302, a switch 303, a battery clip 304, and a battery 305. Finally, the advanced ovulation predictor device contains a cap 103 to protect the objective plate 202, hybrid lenses 201, and turn off the switch 303 when engaged with the advanced ovulation predictor device body.

The switch 303 may be placed anywhere in the advanced ovulation predictor device 100 and may have any type of switch 303 known in the art, including an on-off mechanical switch 303, a spring loaded switch 303, a momentary-on push button 303, a magnetic reed switch 303, an electronic field activated switch and the like. Another embodiment of this invention is one in which the advanced ovulation predictor device cap 103 optionally attaches to the advanced ovulation predictor body.

In another embodiment, there are one or more hybrid lenses 201. The hybrid lenses 201 are made from any material known on the art, including glass, plastics or resins. The lens shape is selected from concave, convex, plano-convex, spherical, aspheric, non-linear, adaptive, and complex combinations of the forgoing. In a preferred embodiment, the one or more lenses 201 are hybrid lenses.

Therefore, the present invention succeeds in conferring the following, and other not mentioned, desirable and useful benefits and objectives.

It is an object of the present invention to provide a portable hand-held advanced ovulation predictor device.

It is another object of the present invention to provide an enlarged optical field of view, a sharper image, and position dependent focus.

Yet another object of the present invention is to provide an automatic light activation switch.

Still another object of the present invention is to provide an advanced ovulation predictor device that includes a battery that is capable of providing long-term and reliable power by eliminating battery contacts, incorporating the contacts into the battery casing.

Yet another object of the present invention is to provide a device that may be able to be powered by DC or AC currents, through storage of mechanical actions converted to an electrical energy, via a piezoelectric source, by capturing electrical fields present in the environment, or by utilizing panels that convert solar light and storing it.

Still another object of the present invention is to provide a method of determining ovulation in a female from a specimen of her bodily fluid, such as saliva, allowing it to dry, and observing a particular “arborization” pattern utilizing the ovulation predictor device 100 described herein.

### BRIEF DESCRIPTION OF THE DRAWINGS

FIG. 1 is a side exploded view of an embodiment of the present invention.

### DETAILED DESCRIPTION OF THE PREFERRED EMBODIMENT

The preferred embodiments of the present invention will now be described with reference to the drawings.

Reference will now be made in detail to the embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

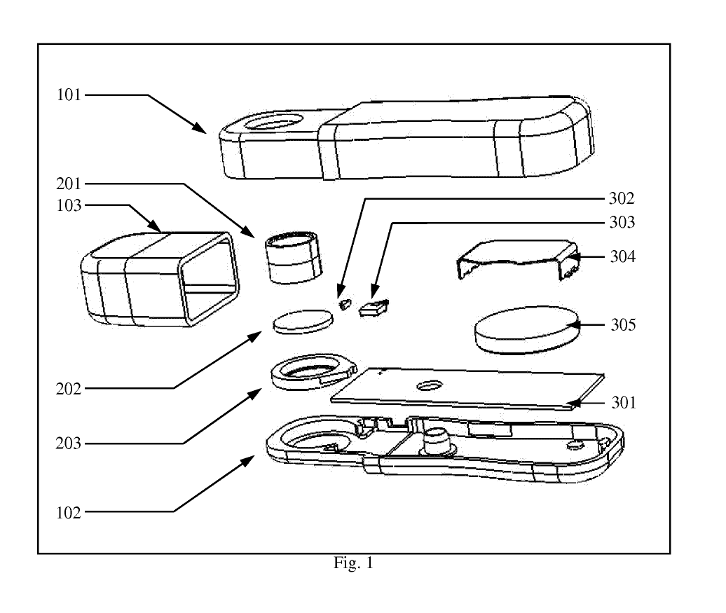

FIG. 1 is a side exploded view of a preferred embodiment of the present invention. Shown are the advanced ovulation predictor device body top 101, the advanced ovulation predictor device body bottom 102, and the advanced ovulation predictor device cap 103. Further the optical subassembly containing one or more hybrid lenses 201, an objective plate 202, and a light pipe 203 are held within the advanced ovulation predictor device body. Yet further the electronics assembly consisting of the printed circuit board 301, a light emitting diode 302, a switch 303, a battery clip 304, and a battery 304 are also held within the advanced ovulation predictor device body.

The switch 303 may be placed anywhere in the advanced ovulation predictor device 100 and may have any type of switch 303 known in the art, including an on-off mechanical switch 303, a spring loaded switch 303, a momentary-on push button 303, a magnetic reed switch 303, an electronic field activated switch, and the like. Another embodiment of this invention is one in which the advanced ovulation predictor device cap 103 optionally attaches to the advanced ovulation predictor body.

The light pipe 203 is a transparent device that transports light from one location to another, acting as an optical waveguide, by relying on internal reflections caused by differences in material index of refraction and/or reflective surfaces. The design of the light pipe allows it to accept emitted light from the light emitting diode, 302, and direct light uniformly around the objective plate, 202, causing the sample to be illuminated in dark field. Dark field illumination enhances the contrast ratio between the background and the sample by scattering the light from the sample instead of absorbing the light.

In yet another embodiment, the light pipe additionally polarizes the light creating alternatively high contrast ratios between the background and the sample by rotation of the observed light wavefront through the sample.

In another embodiment, there are one or more hybrid lenses. The hybrid lenses are made from any material known on the art, including glass, plastics or resins. Simple lenses, such as convex, concave, planar or spherical lenses exhibit spherical aberrations and chromatic aberrations. Aspherical lenses eliminate spherical aberrations, improve chromatic aberrations, but cannot eliminate them. A hybrid lens, incorporating both refractive and diffractive characteristics, eliminates both spherical and chromatic aberrations. The lens shape is selected from concave, convex, plano-convex, spherical, aspheric, non-linear, adaptive, and complex combinations of the forgoing. Spherical aberration is caused by most lenses that are constructed from sections of a sphere. A simple convex lens is a section of a much larger sphere. Light entering the lens along the center axis is focused differently than light entering along the edges. So, viewing an image through a spherical lens will be sharp in the center and increasingly blurry as the distance increases form the center axis. An aspherical lens corrects for this by creating a complex lens surface calculated mathematically to focus to the same spot regardless of where the light enters the lens. Chromatic aberration (also longitudinal aberration) is caused by the fact that light of differing wavelengths focuses at different spots. Thus, if an aspherical lens that focuses blue light perfectly, with no spherical aberrations, the red light entering the lens will not focus at the same spot as the blue light. This is corrected by a diffractive surface that correct for chromatic aberration. A lens that incorporates both features simultaneously is called a hybrid lens. Prior art was not able to utilize hybrid lenses because economical manufacturing techniques were not available. In a preferred embodiment, the one or more lenses, 201, are hybrid lenses.

In the preferred embodiment, the objective plate, 202, is made of optical glass, borosilicate glass, optically clear plastic, or other materials known in the art. The objective plate, 202, has one surface accessible from the exterior of the device, allowing the sample to dehydrate faster than samples processed in prior art.

In yet another embodiment, the hybrid lens or lenses and the objective plate are combined into a single unit, further reducing manufacturing and assembly complexity.

In preferred embodiments, the light, 302, source is a Light Emitting Diode. The light emitting diode wavelength is chosen to optimize the contrast ratio between the background and sample, by matching the characteristics of the lens design, and for saliva said wavelength is between 510 nm and 570 nm. A hybrid lens enlarges the optical field, reduces aberration and blurring of the image as compared to a spherical lens, and incorporates the features of multi-lens systems to reduce the manufacturing and assembly complexities associated with individual lenses.

In the advanced ovulation predictor device electronics assembly the battery, 305, has one or more contacts, preferably a positive and a negative contact, incorporated as part of the battery casing.

In another embodiment, the battery is a surface mount battery with one or more contacts formed to facilitate surface mount soldering directly to the printed circuit board.

In yet another embodiment, the battery is a through-hole battery with one or more contacts formed to solder directly to the printed circuit board utilizing conductive holes and circuit paths as part of the printed circuit board.

The power supplied to the invention may also be generated by any of the following: batteries; rechargeable batteries; solar panels; piezoelectric actuators or sensors; storage of mechanical actions converted to electrical energy by passing a magnet through a conductive coil, said mechanical activation by moving, shaking or cranking; harvesting power from fields generated by power lines or radio transmitters; or capturing time varying fields or physically passing the device through static fields to capture and store energy.

The advanced ovulation predictor device 100 and the body 101 and 102 may have a prismatic shape with ergonomic features, but may have any other shape, including round, tubular or cylindrical, spherical shape, square, triangular, rectangular, complex, and the like. The body 101 and 102 may have a smooth, textured, or an uneven surface. In one embodiment the body 101 and 102 may have may have ribs, finger indentations, or other shapes or indentations which are useful in achieving a good grip and to hold the advanced ovulation predictor device 100.

The advanced ovulation predictor device body, 101 and 102, may be of any length, width, or height. In a preferred embodiment the ovulation predictor device 100 fits in the palm of a human hand or in a woman's purse. In one embodiment, the dimensions are approximately 0.9 inches wide by 0.5 inches thick by 3.0 inches long. In another embodiment, the dimensions may have different ranges as in prismatic or irregular shapes. One dimension may be equal to or greater than or less than the other dimensions. In other embodiments, the length may be about 1.0 inches to about 6.0 inches.

In a preferred embodiment the specimen is saliva. In another preferred embodiment, the user views the arborization pattern created by crystals formed by the dried specimen when using the advanced ovulation predictor device 100.

The advanced ovulation predictor device 100 optionally contains a carrying case designed to protect the device when carrying the device, for example in a purse, pocket, or luggage. In a preferred embodiment, the advanced ovulation predictor device is a kit comprising the ovulation prediction device 100, a lens cleaning cloth, instructions, and instructional media. The instructional media may be written or in electronic format or may utilize more than one means of communication of instructions and educational materials. These instructions and education materials may be in one or more languages. The advanced ovulation predictor device 100 may be supplied in a kit comprising the ovulation predictor device of claim, instructions on how to use the device and, optionally a carrying case. A kit may also comprise the ovulation predictor device, instructions on how to use the device, a carrying case and a cloth with which to clean the objective plate. In a most preferred embodiment, the kit comprises the ovulation predictor device, instructions for use, an interactive instructional media device such as a memory stick, a discrete carrying case, a microfiber cleaning cloth, and a fertility chart. The instructions may contain information on how to utilize the ovulation predictor device 100 and other useful or educational material.

A preferred aspect of the invention is a method of predicting ovulation in an animal, utilizing the ovulation predictor device 100 described herein, said method comprising:
1. placing a specimen on the objective plate;
2. permitting the specimen to dry; and
3. viewing the crystals produced from said dried specimen.

Although any bodily fluid can be utilized as a specimen for this method of detection, such as saliva, tears, blood, vaginal secretions, or sweat, in a preferred embodiment, the specimen is a sample of saliva. In another preferred embodiment, the animal is a human female.

In a preferred embodiment, the invention consists of a kit which contains the advanced ovulation predictor device, instructions for use, a lens cleaning cloth, instructional media, a fertility chart, and optionally a carrying case for the device.

While certain novel features of the present invention have been shown and described, it will be understood that various omissions, substitutions and changes in the forms and details of the device illustrated and in its operation can be made by those skilled in the art without departing from the spirit of the invention.

I claim:

1. An advanced ovulation predictor device, comprising:
a device casing;
an optical subassembly within the device casing;
an electronics assembly within the device casing; and
a light source within the device casing, electrically connected to the electronics assembly, and adjacent to the optical subassembly.

2. The advanced ovulation predictor device of claim 1, wherein said optical subassembly comprises
an objective plate;
a light pipe adjacent to the objective plate and to the light source; and
a hybrid lens adjacent to the objective plate.

3. The advanced ovulation predictor device of claim 1, wherein said electronics assembly comprises
a circuit board;
a switch; and
a power supply.

4. The advanced ovulation predictor device of claim 2, wherein said hybrid lens comprises
an aspherical lens; and
a diffractive surface to correct for chromatic abberation.

5. The advanced ovulation predictor device of claim 4, wherein said hybrid lens has an enlarged optical field, reduces aberration and blurring of an image viewed through the hybrid lens, and reduces manufacturing and assembly complexity as compared to a spherical or aspherical lens.

6. The advanced ovulation predictor device of claim 2, wherein said hybrid lens forms a focal length that is about 0.12 inches.

7. The advanced ovulation predictor device of claim 2, wherein the light pipe comprises a transparent body adjacent to the light source and to the objective plate.

8. The advanced ovulation predictor device of claim 7, wherein the light pipe comprises a circular body surrounding the objective plate.

9. The advanced ovulation predictor device of claim 7, wherein the light pipe comprises a polarizer.

10. The advanced ovulation predictor device of claim 2, wherein said objective plate has a planar surface accessible from the exterior of the casing.

11. The advanced ovulation predictor device of claim 10, wherein said objective plate is comprised of a transparent material.

12. The advanced ovulation predictor device of claim 3, wherein the power supply is selected from one of the following:
a battery
a rechargeable battery;
a solar panel;
a piezoelectric actuator or sensor;
storage of mechanical actions converted to electrical energy by passing a magnet through a conductive coil, said mechanical activation by moving, shaking or cranking;
harvesting power from fields generated by power lines or radio transmitters; or
capturing time varying fields or physically passing the predictor device through static fields to capture and store energy.

13. The advanced ovulation predictor device of claim 1, wherein the light source is a light emitting diode.

14. The light emitting diode of claim 13, wherein said light emitting diode emits a wavelength of light in the visible spectrum.

15. The light emitting diode of claim 14, wherein said light emitting diode emits a light with a wavelength between 510 nm and 570 nm.

16. The advanced ovulation predictor device of claim 1, wherein said device casing is rectangular with an ergonomically curved shape.
